(19) United States
(12) Patent Application Publication
(10) Pub. No.: US 2013/0133665 A1
(43) Pub. Date: May 30, 2013

(54) Full Genital Protective Barrier Garment Device

(71) Applicant: Anita Lynn Waller, Colorado Springs, CO (US)

(72) Inventor: Anita Lynn Waller, Colorado Springs, CO (US)

(21) Appl. No.: 13/304,594

(22) Filed: Nov. 25, 2011

(51) Int. Cl. A61F 6/06; A41B 9/00; A61F 6/02

(52) U.S. Cl. 128/830; 128/842; 2/400

(57) ABSTRACT

A practical, disposable full genital barrier/shield prophylactic undergarment that is gender specific with at least one inwardly or outwardly expandable and collapsible orifice covering for safe sex protection from body fluids, pathogens and parasites for a wearer and a partner which includes integral and multiple sensory, textural, visual and/or body enhancement components along with herbal, chemical and/or natural elements for increased stimulation while providing comfort, portability, style and size options in an undergarment device.

### BACKGROUND OF THE INVENTION

There is increased concern among people who are sexually active about exposing their partner to or contracting diseases spread from contact with body fluids, germs, viruses, bacteria, micro-organisms, fungi, pathogens, parasites and other contaminants which may be present in or on surrounding genitalia or other body parts during sexual intercourse or foreplay. While a male or female condom does offer some degree of protection; a large surface area surrounding male or female genitalia and lower body parts is still exposed when using standard condoms. There exists a need for a protective undergarment device which covers a larger genital surface area, and that shields the wearers and their partners from sexually transmitted diseases, (STD's), etc., while they are sexually engaged; and which simultaneously allows for penetration of one or more body orifices without breaking or disrupting the protective barrier/shield. There have been other prior patents that have incorporated the use of shields and prophylactic condoms in the undergarment designs. However, no other prior designs have incorporated as many new, innovative, and unique integral elements that simultaneously provide protection, enhanced sensory stimulation, convenience, economy, attractiveness, and enjoyment for both partners, and both genders, as the instant invention. U.S. Pat. No. 6,694,980 B2 by Anderson shows a system that incorporates separate components which include a garment, condom, sexual aids, and an informational brochure in pockets on the garment. U.S. Pat. No. 5,638,829 by Najor shows a belted garment with an integral condom element that can be inverted for use by a male or a female. U.S. Pat. No. 5,596,997 by Abadi shows a female panty with a removable crotch portion that has an integral condom attached. U.S. Pat. No. 5,535,757 by Fleming, Jr. shows a panty with a crotch that has an opening to access an attached condom. U.S. Pat. No. 5,269,320 by Hunnicutt shows a device and an undergarment with integral condoms over the vaginal and anal areas. U.S. Pat. No. 5,181,527 by Dorsey et al. shows a garment with integral condom and removable tabs that help enclose the condom in a pouch. U.S. Pat. No. 4,981,147 by Barnett shows a garment with an integrally attached condom that can be inverted for male or female use. U.S. Pat. No. 4,862,901 by Green shows a garment with integral condom and integral texture on the crotch to be worn by a female.

### FIELD OF INVENTION

This present invention relates to the fields of undergarments and male condoms and female condoms, unisex condoms, protective barrier shields, and physical birth control devices.

### SUMMARY OF THE INVENTION

This present invention is directed to a full genital protective barrier undergarment device that allows the user to fully cover and protect his or her genitalia during sexual intercourse or activity and provide protection from and against contamination caused by contact of bodily fluids, viruses, bacteria, fungi, micro-organisms, STD's, germs, and other pathogens; with protection being restricted to and from the area covered by said garment device. The device also simultaneously allows for the sexual activity penetration of one or more body orifices without breaking the protective shield barrier of said device, by use of both an integrally and permanently attached male prophylactic condom, plus a unisex anal prophylactic condom on a garment device, or by use of both an integrally and permanently attached female prophylactic condom, plus a unisex anal prophylactic condom on an undergarment device. The condom elements may have, all or in part, a smooth or a textured surface. The condoms may be pleated, folded, bellowed, scrunched, ringed, or telescoped to all allow them to be flattened and compressed when not in the extended/expanded state. The undergarment device may also provide an optional chemical, as well as the inherent physical, spermicidal barrier as an added layer of protection against unwanted pregnancy. The device provides enhanced visual and physical sexual stimulation and enjoyment for wearers and their partners from the integral textured, flavored, scented, printed, tinted, colored, and/or decorated exterior and or interior surfaces of said undergarment device. It may also contain an integral or surface lubricant, (liquid, gel, or powder), and may also have a sensation enhancement element that is integral. The garment, in a preferred embodiment, incorporates herbal, natural and/or chemical sexual organ stimulant elements that are integral within, or applied to a surface of the garment and are strategically positioned on the garment so that said elements are proximal to a wearer's or a partner's sexual organs and/ or erogenous zones. It has stretchy, flexible, comfortable, optimal temperature retaining material/substance/fabric proximal to the scrotum area. The garment is easy to put on and take off, and it is disposable. The undergarment, in the preferred embodiment, may be comprised, all or in part, of liquid impermeable, biodegradable and inexpensive material substance, such as, but not limited to: latex, polyurethane, nitrile, rubber, vinyl, sheepskin, cellulose, plastic, silicon, spandex, cotton, cloth, rayon, nylon, viscose, vegan substance, and any other material that may be known now or used in the future by someone skilled in the art of making condoms, surgical gloves, and/or disposable gloves commonly used for medical purposes, and undergarments. In addition, the undergarment device may be comprised, all or in part, of one or more layers of substance material to provide protection and comfort. In the preferred embodiment, the device is transparent or translucent to effectuate and simulate the appearance of a nude body while wearing the garment device. The device may have integral elements which may enhance the appearance of the wearer's body, such as contouring, molding and padding strategically positioned on the buttocks area of the device. The undergarment device is comprised of component elements appropriate for accommodating either a male's or a female's genitalia, in which one embodiment is distinguished as a “male” garment, and the other embodiment is distinguished as a “female” garment. Both the male and female embodiments are to be considered part of, and integral to the scope and intent, and idea of said undergarment device, and either garment may be worn by either a male or a female person if they so choose.

### BRIEF DESCRIPTION OF THE DRAWINGS

The foregoing features of this invention, as well as the invention itself, may be more fully understood from the following description of the drawings in which:

FIG. 1 is a front view of a female full genitalia protective barrier garment device.

FIG. 2 is a front view of a male full genitalia protective barrier garment device.

FIG. 3 is an enlarged front view of a female garment device in which the integrally attached crotch area (1) has integral stretchy, vulva/vestibule covering (2) with stretchable and inwardly expanding rings, bellows, pleats, or folds, etc. (3) and a center reservoir area (4). The garment device has snug leg bands (5), a snug waistband (6), and may have areas on the sides of the device (7) or elsewhere to accommodate elements for easy donning or removal of device.

FIG. 4 is an enlarged front view of a male garment device. The upper crotch area (8) has integral stretchy penis covering (9) with stretchable and outwardly expandable rings, bellows, folds, or pleats, etc. (10), and a center reservoir tip (11). The male garment device also has a lower crotch area (12) with integral scrotum/testicle covering (13). The male garment device has snug leg bands (14) , snug waistband (15), and may have areas on the sides (16) or elsewhere to accommodate elements for easy donning or removal of device.

FIG. 5 is a bottom angled view of a female device. In the preferred embodiment, stretchable inwardly expanding covering is over the vulva/vestibule area (17). In another embodiment, a similar inwardly expanding covering is over the rectal/anal orifice (18) to facilitate penetration.

FIG. 6 is a bottom angled view of a male device. In the preferred embodiment, the stretchable, outwardly expandable covering is over the penis area (19). In another embodiment, a comfortable, optimal temperature retaining substance/material/fabric covers the scrotum/testicle area (20). In another embodiment, stretchable, inwardly expandable covering is also over the rectal/ anal orifice (21) to facilitate penetration.

FIG. 7 is an anterior view of a female wearing a female garment device. In the preferred embodiment, the crotch of the garment has the stretchable, inwardly expandable covering over the vulva/vestibule area to facilitate penetration into the vaginal cavity (22).

FIG. 8 is a posterior view of a female wearing a female garment device in which the crotch of the garment extends past the rectal/anal area, and may have a seam element between the buttock cheeks (23) to define and accentuate the contours of the buttocks. The device may also have integral molding or padded contouring of the buttocks area (24) of the device to facilitate the appearance of larger buttocks of the wearer.

FIG. 9 is an anterior view of a male wearing a male garment device. In the preferred embodiment, the crotch of the garment has a stretchable, expandable covering over the penis (25), and stretch soft comfort and/or moisture absorption covering on testicle/scrotum area (26).

FIG. 10 is a posterior view of a male wearing a male garment device in which the crotch of the garment extends past the rectal/anal area and may have a seam element between the buttock cheeks (27) to define and accentuate the contours of the buttocks. The device may also have additional integral molding and/or padded contouring in the buttocks area to facilitate the appearance of larger buttocks of the wearer.

FIG. 11 is a front view of a female wearing a female garment device in the preferred “boy cuff shorts” embodiment.

FIG. 12 is a front view of a male wearing a male device in the preferred “boy cuff shorts” embodiment.

FIG. 13 is a diagram side view of a female body showing the erogenous zones: the “U-spot” (28), which is proximal to the urethral orifice, the clitoris (29), the “G-spot” (30) on the vaginal wall, and the “A-spot” (31), which is proximal to the cervix.

FIG. 14 is a side cut away view of a female garment device in which the interior surface of the crotch has texture proximal to a wearer's clitoris (32), the “U-spot” (33) of the urethral orifice, the “G-spot” (34) on the vagina, the “A-spot” (35) of the cervix, and the rectum/anus (36).

FIG. 15 is a cut-away side view of a female wearing a female garment device. The penis (37) is pushed against the vestibule/vulva covering (38), into the vaginal cavity (39).

FIG. 16 is a cut-away side view of a female wearing a female garment device in which a penis (40) is pushed against the vulva/vestibule covering (41). This female condom component (42) expands and fully encases penis within the vaginal cavity (43).

FIG. 17 is a side view of a female garment device in which the exterior surfaces of the female condom component has texture, and/or chemical and/or herbal sexual stimulant element on the side walls (44) and at the reservoir tip (45). There may also be a spermicidal barrier (46) integral or on the exterior, and/or interior surface of device.

FIG. 18 is a side view of a female garment device in which the interior surface area proximal to the wearer's “U-spot”(47), clitoris (48), “G-spot” (49), and “A-spot” (50), have a natural, chemical, and/or herbal sexual stimulant element integral within, or applied to the interior surface of said device which contacts the wearer's skin

FIG. 19 is a side view of a male wearing a male garment device. In this preferred embodiment, the stretchy male condom element (51) covers the penis, and has stretchy expandable rings, bellows, folds, pleats, etc. (52) and a reservoir tip (53). The lower crotch area (54) has comfortable, optimal temperature retaining material/substance/fabric element over the scrotum area (55).

FIG. 20 is a side view of a male wearing a male garment device in which the covering is stretched and expanded (56) to accommodate an erection.

FIG. 21 is a side view of a male wearing a male garment device in which the garment fully covers the genital area (57). The male condom element fully encases the erect penis, and has texture, and/or herbal and or chemical sexual stimulant elements on the exterior surface (58) and on the reservoir tip (59). Leg bands (60) help keep body fluids inside the garment.

FIG. 22 is a cut-away magnified side view of a male wearing said device in which the exterior surface may have a smooth, textured, natural, herbal, and/or chemical sexual stimulant element (61) proximal to a woman's “G-spot” during intercourse. The device may have interior (62) and/or exterior (63) texture anywhere along the shaft. The device has a spermicidal element on either surface (64), and a reservoir tip (65).

FIG. 23a is a front view of a female wearing a female garment device in which the garment has just been donned; and the garment is still mostly rolled (66).

FIG. 23b is a front view of a female wearing a device which has been partially unrolled (67).

FIG. 23c is a front view of a female wearing a device which is completely unrolled (68).

FIG. 24 is a front angled view of a female wearing a garment device in which alternate securing or removing elements may be located at sides of garment (69) or at different locations.

FIG. 25 is a front angled view of a female wearing garment device which has a perforated tear off (70) side element integral to the device.

FIG. 26 is a front angled view of a female wearing a device with side zipper (71) elements.

FIG. 27 is a front angled view of a female wearing a device with side snaps (72) elements.

FIG. 28 is a front angled view of a female wearing a device with side press and seal (73) elements.

FIG. 29 is a front angled view of a female wearing a device which has sticky adhesion or Velcro type side elements (74).

FIG. 30 is a magnified cross section side view of a male or female device which may consist all or in part of substance material consisting of one layer (75) thickness with integral barrier element, exterior surface, and interior surface integral within one layer thickness.

FIG. 31 is a magnified cross section side view of a male or female undergarment device which may consist of an exterior layer (76) which may be decorated, flavored, lubricated, scented, and/or textured, and have integral chemical or herbal element; and a interior layer (77) which may have the barrier and/or comfort elements, and contacts the skin

FIG. 32 is a magnified cross section side view of a male or female undergarment device which may consist of an exterior layer with integral and unique elements mentioned previously (78), a middle layer which may have a protective barrier (79), and an interior comfort layer (80) that contacts skin

FIG. 33 is a magnified cross section side view of a male or female device which may consist of an exterior protective barrier layer (81), and an inner comfort layer (82). Note that many configurations of layers and elements are within the scope of the invention. Not all of them have been illustrated here.

FIG. 34 is a front view of a female wearing a female garment device which has been decorated on the exterior surface (83), and may have integral flavors, scents, and textures.

FIG. 35 is a front view of a male wearing a male garment device which has been decorated on the exterior surface (84) and may have integral flavors, scents, and textures.

FIG. 36 is a front view of a modified thong female garment device.

FIG. 37 is a front view of a “boy cuff shorts” style female garment device.

FIG. 38 is a front view of a bike shorts style female garment device.

FIG. 39 is a front view of a hi-rise style female garment device.

FIG. 40 is a front view of a bikini male garment device.

FIG. 41 is a front view of a bike shorts style male garment device.

FIG. 42 is a front angled view of a female garment device which has been decorated and which has the integral modified crotch genital covering.

FIGS. 43a-e display an assortment of designs with corresponding flavors and scents that may be integral to the devices. Examples include, but are in no way limited to: (a) vanilla flavor and scent, (b) apple, (c) chocolate, (d) strawberry, (e) peppermint.

FIG. 44 is a front view of a male garment device which has the integral modified crotch genital covering.

FIG. 45 is a magnified cross section side view of a textured surface for a male or female device which has a prickly surface (85).

FIG. 46 is a magnified cross section side view of a textured surface for a male or female device which has a smooth rounded bumpy surface (86).

FIG. 47 is a top view of a container suitable for bulk/multiple (87) packaging of the garment devices.

FIG. 48 is a front view of an individual packaging of male or female garment device which has a perforated and tear open element (88).

### DETAILED DESCRIPTION OF THE INVENTION

Specifications for full genital male or female protective barrier/shield undergarment device with a flexible barrier/shield that protects the wearer's full genital area from sexually transmitted diseases, (STD's), bacteria, viruses, body fluids, parasites, micro-organisms, pathogens, fungi, germs, and other contaminants which may be present during sexual activity. The device facilitates protected sexual penetration of one or more body orifices by employing the use of one or more permanently attached liquid impermeable, and tear resistant prophylactic condom elements which are integral to the undergarment device. The device also provides protection for the wearer's partner by shielding him or her from the contaminants that are essentially being blocked by the barrier device. The device provides protection from and against contaminants on the areas covered by, and enclosed within the garment device.

The undergarment device, in preferred embodiment, is a “boy cuff shorts” style panty or brief, comprised (all or in part) of liquid impermeable material substance that is transparent or translucent, and which has snugly fitting leg bands, and/or “boy cuff shorts” style leg cuff bands, and has a snugly fitting waistband to prevent fluid leakage. The device may be comprised (all or in part) of, and in any combination of, one or more layers of substance material such as latex, rubber, cellulose, cloth, cotton, or any other substances used by those skilled in the art of making sterile surgical or disposable gloves, condoms, and undergarments. The undergarment device has interior and exterior surfaces that may be textured, contoured, decorated, flavored, scented, and treated with herbs, natural and/or chemical elements positioned specifically proximal to erogenous zones and other sensitive areas, to provide increased sexual arousal; and to provide visual and physical stimulation for the wearer and his or her partner. The use of natural, chemical, and/or herbal elements which are integral to the undergarment, and specifically positioned proximal to erogenous zones is a unique and important concept of this instant invention. The undergarment device may contain integral spermicidal elements for protection against unwanted pregnancy, as well as integral lubrication elements. The device has elements that may enhance the wearer's body appearance, particularly the buttocks area. In one embodiment, the scrotum area of the male garment device has soft, comfortable, stretchy, optimal temperature retaining substance/material/fabric element, as well as a moisture wicking element. The device is sterile, thin, lightweight, body conforming, or in other embodiments, loose fitting. The undergarment device is comfortable, attractive, and easy to put on and remove. In the preferred embodiment, it is portable, disposable, bio-degradable, and economical, and packaged singly or in bulk.

The illustrations of the garment device and the exemplary embodiments are to help convey the basic concept of this immediate invention and to help one visualize some of the variations and configurations that are within the scope of the idea. These embodiments are not intended to limit the scope, intent, or spirit of the invention in any way.

Because of the increase of sexually transmitted diseases, (STD's), worldwide; there is a need for a device to help limit the spread of disease through contact of body fluids, germs, pathogens, micro-organisms, viruses, bacteria and parasites during sexual activity. Male and female condoms provide some protection from diseases and for birth control. However, condoms only protect the immediate area covered by the condoms. There is a need, therefore, for an undergarment device that covers a larger genital and lower torso body surface area to help protect against contamination from exposure to sexually transmitted diseases, (STD's), body fluids, pathogens, viruses, germs, bacteria, fungi, and parasites during sexual activity and contact. The undergarment device provides an added layer of safe sex protection. Because of the new, innovative, and unique elements which are integral to this instant invention, it may be the more preferred and desirable protection choice for safe sex. It does not have the appearance or the stigma of an adult diaper protective garment.

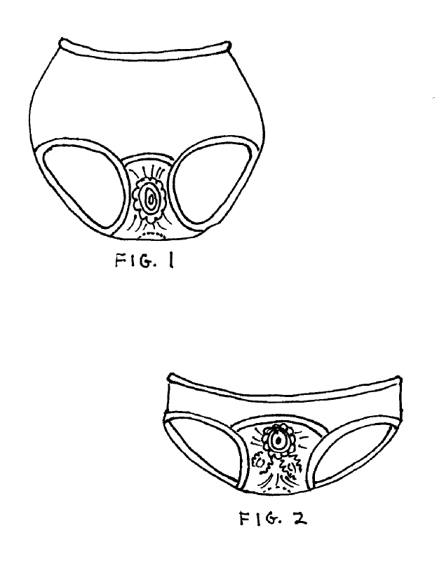

The full genital protective shield garment device comes in a female (FIG. 1) and male (FIG. 2) embodiment configurations. In preferred embodiments, it has an integral, thin, flexible liquid impermeable barrier shield composed of latex, polyurethane, sheepskin, rubber, nitrile, or other substance material similar in content to condoms and/or disposable, sterile surgical gloves. The garment device may also consist of one or more layers of various, above mentioned, substance material, which may vary from one area of the garment to the other. In addition, the garment device may consist of other substance material such as: cotton, nylon, polyester, rayon, or other material used by a person skilled in the art of making undergarments. Such undergarment fabric/substance/material would be used on various areas of the garment to provide comfort, sweat absorption, body temperature control, and to make the device aesthetically pleasing, and provide physical stimulation. The panty or brief itself, which includes the modified crotch portion, the leg bands, a waistband, and one or more condom elements are all permanently attached and integral to the basic embodiment of the full genital protective shield undergarment device, and said device is intended to be removed and disposed of after use.

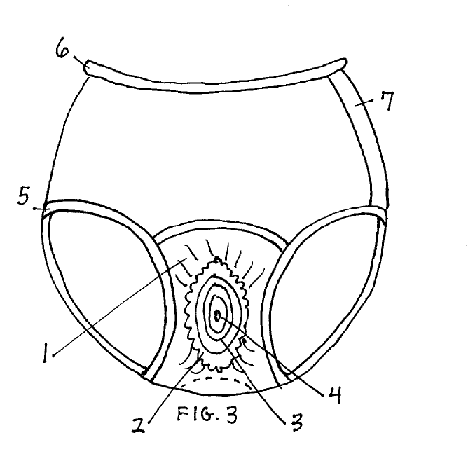

In the preferred embodiment of a female garment device (FIG. 3); the modified crotch area (1), has integral pubic/vulva covering (2), with stretchable and inwardly expandable/extendable substance material rings, bellows, folds, or pleats, etc., of covering (3) and a center reservoir tip (4) proximal to the vaginal vestibule. The garment has snugly fitting leg bands or cuff bands (5), and a snugly fitting waistband (6), to help prevent leakage of bodily fluids, and to help secure garment to the body. The garment may also have side or other area elements (7) to facilitate easy donning or removal of said garment device.

In the preferred embodiment of the male garment device (FIG. 4), the modified upper crotch area (8) has integral penal covering (9) with stretchable and outwardly expanding substance material rings, bellows, pleats, folds, etc., of covering (10) and a reservoir tip (11) proximal to the penis opening. The male garment device also has a lower crotch area (12) with integral scrotum/testicle covering (13) made of stretchy, comfortable substance. The male garment device has snug leg bands (14), and a snug waistband (15). The garment may also have side, (16), or other area, elements to facilitate easy donning or removal of said garment device.

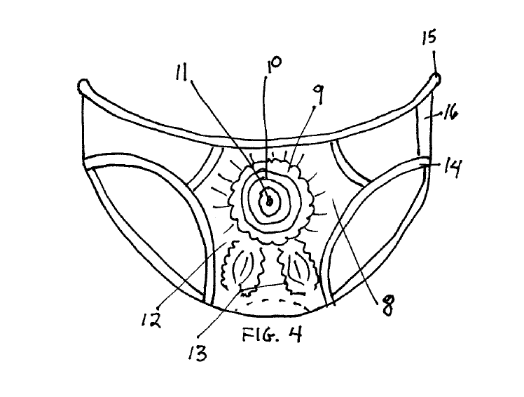

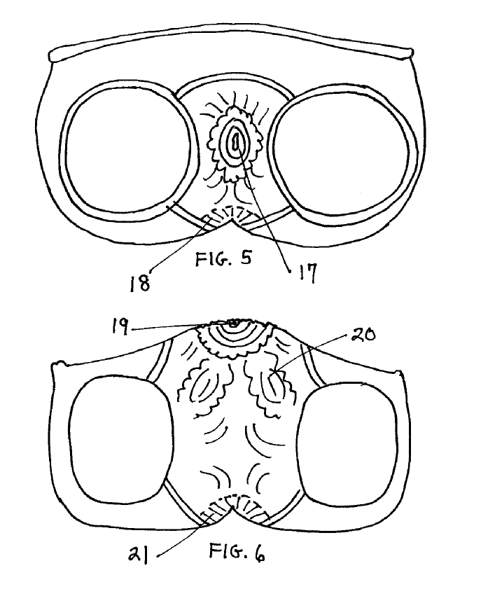

In the preferred embodiment of a female garment device (FIG. 5), the stretchy, inwardly expanding element of the modified crotch area covers the vulva/vaginal vestibule orifice area (17); and comprises the female condom component element of the device. In another embodiment, similar stretchy, inwardly expanding element may also cover the rectal/anal orifice area (18) and comprises a unisex anal condom.

In the preferred embodiment of a male garment device (FIG. 6), the stretchy, outwardly expanding element (19) of the modified upper crotch area covers the penis; and comprises the male condom component element of the device. Comfortable, stretchy, optimal body temperature retention substance/material/fabric covers the scrotum area (20) to help keep the testicles at optimal temperature. In another embodiment, stretchy, inwardly expanding element may cover the rectal/anal orifice (21) and comprises a unisex anal condom. The stretchy inwardly or outwardly expanding/extending condoms can be rolled, telescoped, scrunched, folded, pleated, or otherwise compressed and flattened while in the un-extended, un-expanded state prior to insertion of a member.

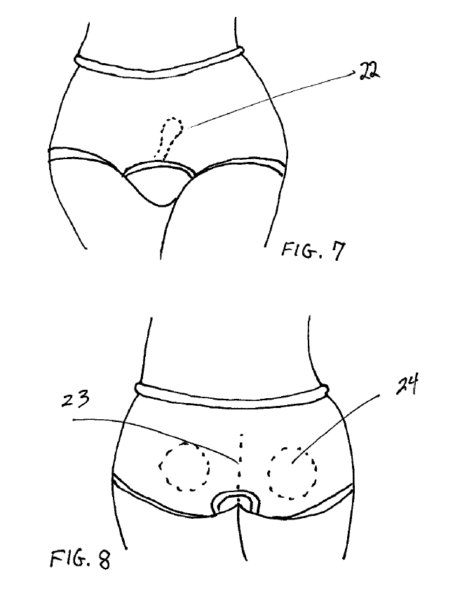

(FIG. 7) illustrates a female wearing a garment device in which the modified crotch begins at the pubic bone area of the body, and covers the vaginal vestibule area with the female condom component, which is pushed into the vaginal cavity (22) during intercourse. In (FIG. 8), the said modified crotch extends past the anal area. In an embodiment, there is a rear seam element (23) to divide the buttock cheeks, and enhance the contour of the buttocks. The device may also have, in some embodiments, integral molding and/or contour padding (24) for enhancement of the appearance of the buttocks, and/or to make the buttocks look larger.

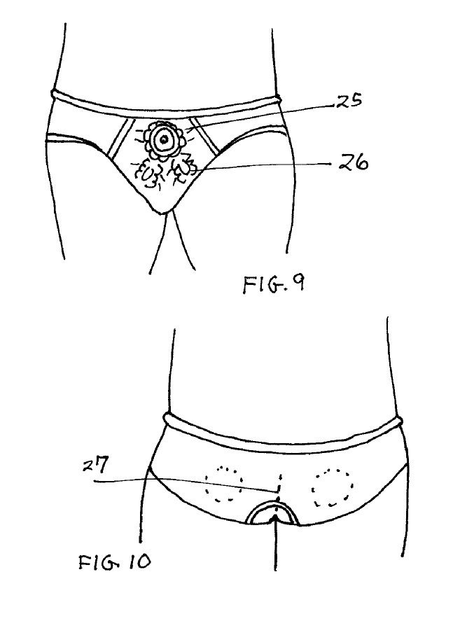

(FIG. 9) illustrates a male wearing a garment device in which the modified crotch covers the penis area (25) and the testicle/scrotum area (26). In (FIG. 10), the said modified crotch extends past the rectal/anal area. In another embodiment, there is a rear seam element (27) to define and enhance the contour of the buttocks, as well as molding, and contour padding of the buttock area to give the appearance of larger buttocks.

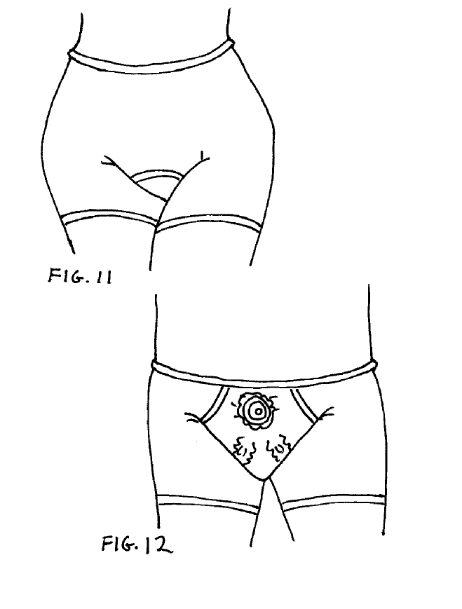

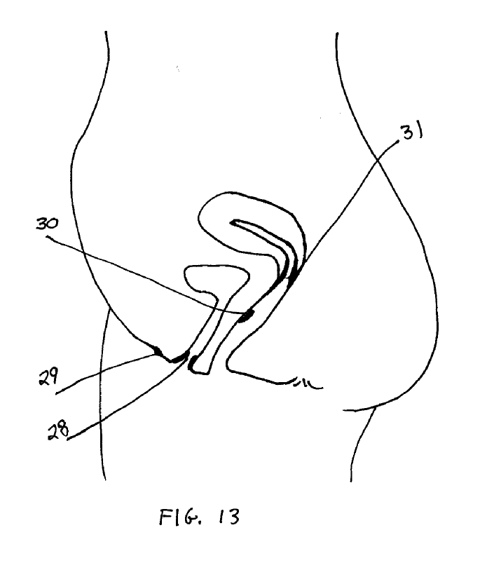

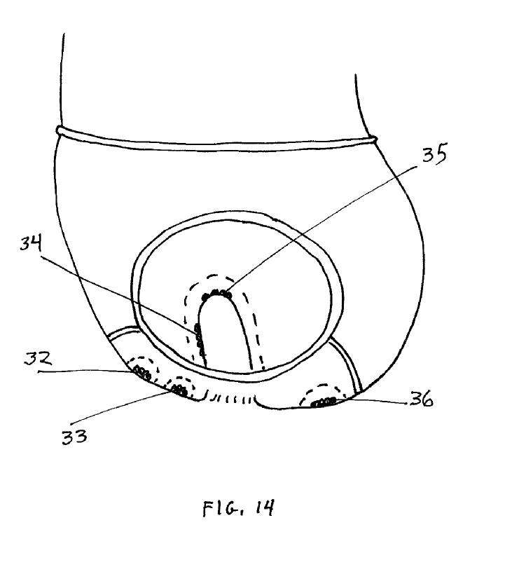

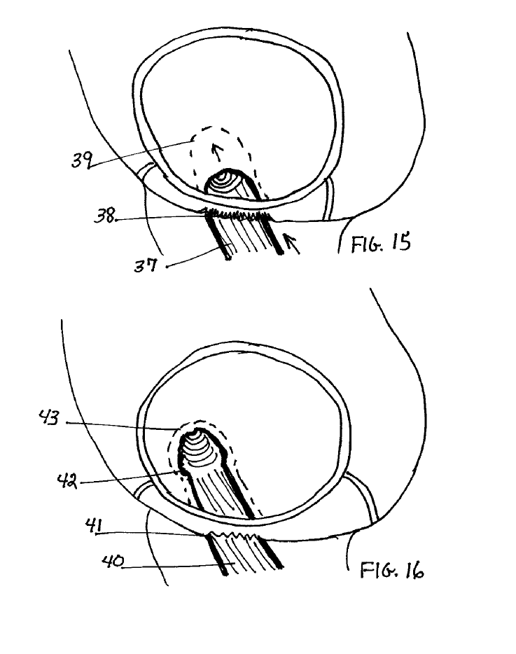

In (FIG. 11), the crotch modification is not immediately noticeable on the female garment; as the modification area is somewhat hidden between the legs. However, in (FIG. 12), the male crotch modification is immediately apparent. The “boy cuff shorts” style is the preferred embodiment for the garment device however, any style may be used. On the diagram in (FIG. 13), one can see the female erogenous zones: the “U-spot” near the urethral orifice (28), the clitoris (29), the “G-spot” on the vaginal wall (30), and the “A-spot” near the cervix (31).

As seen in (FIG. 14), the stretchy, expandable element covers the vaginal vestibule area and inwardly expands into the vaginal cavity. In this embodiment, the modified crotch area has interior surface texture proximal to the clitoris (32), urethra (33), and rectal/anal orifice (36). The interior surface of the female condom component has texture proximal to the “G-spot” (34), and the “A-spot” (35). The interior texture provides enhanced stimulation and pleasure for the wearer of the device. Texture is not limited to the examples given. As illustrated in (Figure #15), the penis (37) pushes against the exterior surface of the stretchy element covering the vaginal vestibule (38) of the female garment device which inwardly expands into the vaginal cavity (39). In the preferred embodiment, as illustrated in (FIG. 16), the penis (40) pushes against the vaginal vestibule covering (41) of a female device, and then becomes fully encased within the stretchy, inwardly expanding female condom element (42), while within the vaginal cavity (43). It should be noted that both partners may wear an undergarment device simultaneously while involved in sexual activity intercourse and/or foreplay.

In another embodiment of the female garment device in (FIG. 17), the female condom portion of the garment which enters the vaginal cavity may have external surface texture elements on the sides (44), and/or on the external reservoir tip surface (45) to facilitate the wearer's partner's penis stimulation and to enhance the partner's pleasure. However, the placement of said exterior texture elements is not limited or restricted to the example given. The condom element may contain lubricating elements that are integral, or applied to the condom surfaces, depending on substances used. The condom element may contain integral spermicidal element within, or on either surface area (46). The spermicidal and lubricant elements may be employed in the male and/or female undergarment devices. In another embodiment (FIG. 18), the interior crotch surface on a female garment device may have natural, chemical and/or herbal sexual stimulant elements which are integral or applied, and are positioned on the crotch and being proximal to the urethra (47) and the clitoris (48) of the wearer. The female condom component interior surface may also have chemical and/or herbal sexual stimulant elements which are integral or applied; and are stationed on the condom and being proximal to the “G-spot” (49) and the “A-spot” (50) of the female wearer. It is to be noted that the use of texture, and/or herbal and or chemical sexual stimulant elements integral within or applied upon the male and/or female undergarment device to facilitate particular heightened stimulation of the noted erogenous zones in particular, and other genital areas in general, on either partner during sexual activity; is a unique and integral concept to this instant invention.

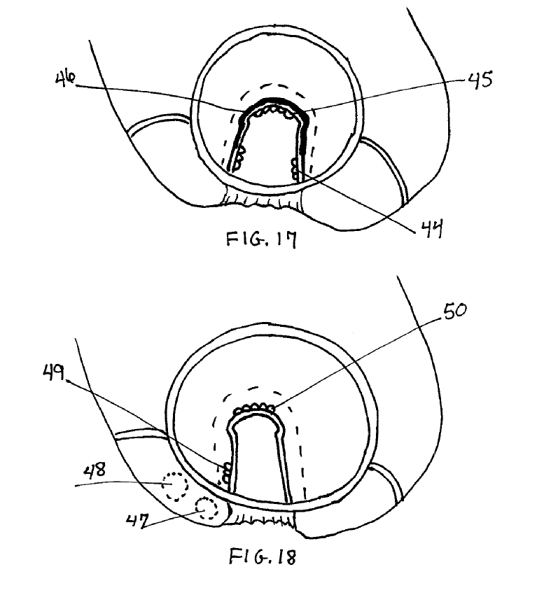

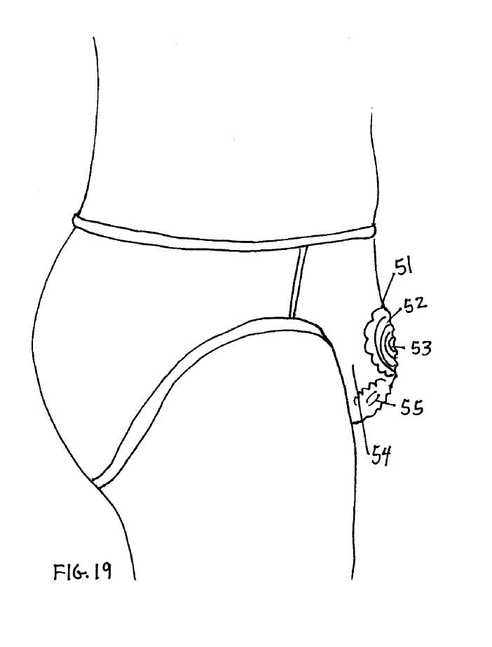

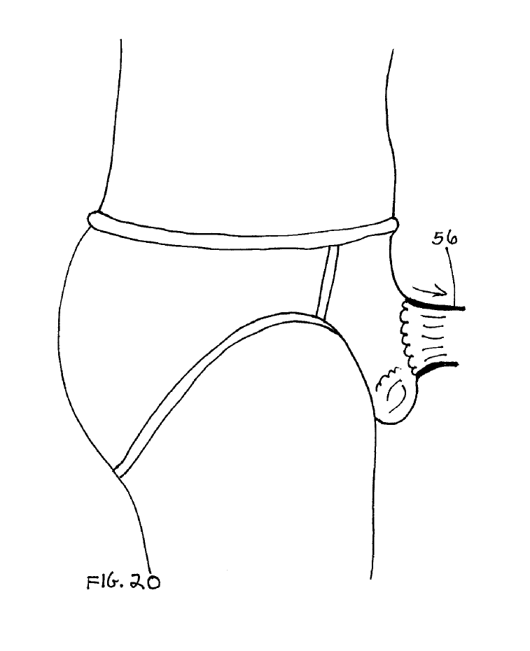

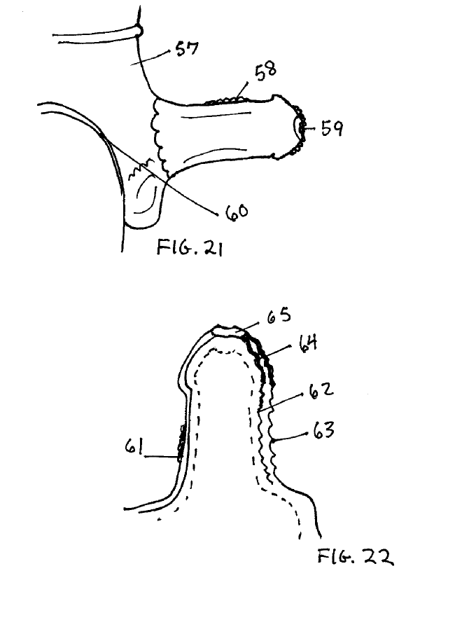

In the preferred embodiment of a male garment (FIG. 19), the modified upper crotch stretchy male condom element (51) covers the penis, and has outwardly expanding/extending substance material element rings, folds, pleats, bellows, etc. (52), and a reservoir tip (53) for semen. In a preferred embodiment, the lower crotch area (54) is modified with comfortable, stretchy, optimal body temperature retaining material substance, or fabric element covering for the scrotum/testicle areas which contacts the wearer's skin (55). This is a unique element which may increase the comfort, and therefore the wearing time of the garment device. (FIG. 20) illustrates how the stretchy outwardly expanding rings, bellows, folds, pleats, etc. (56) may accommodate an erection. As seen in (FIG. 21), the garment device fully covers the male genital area (57), thereby increasing the protection area. The male condom component encases the erect penis. In an embodiment, the male condom has stimulating texture, and/or herbal and or chemical sexual stimulant elements on the exterior side surface (58), and on the reservoir tip surface (59), for partner pleasure. Leg bands (60) help keep fluids inside the garment. As seen in (FIG. 22), the male condom component surfaces of the garment device which cover the penis may be smooth (61), or have internal texture (62) or external texture (63) for the wearer and/or the partner's stimulation. The device may have integral spermicidal barrier (64), and a reservoir tip (65) for ejaculate. The male condom component may have lubricant element which is integral or applied to either surface area. As noted earlier in this text, texture and/or chemical and/or herbal stimulation elements may be incorporated and used anywhere on the internal or external surfaces of the garment device. However, placement proximal to the wearer's or their partner's erogenous zones is the preferred embodiment. This unique idea is integral to the intent of the present invention.

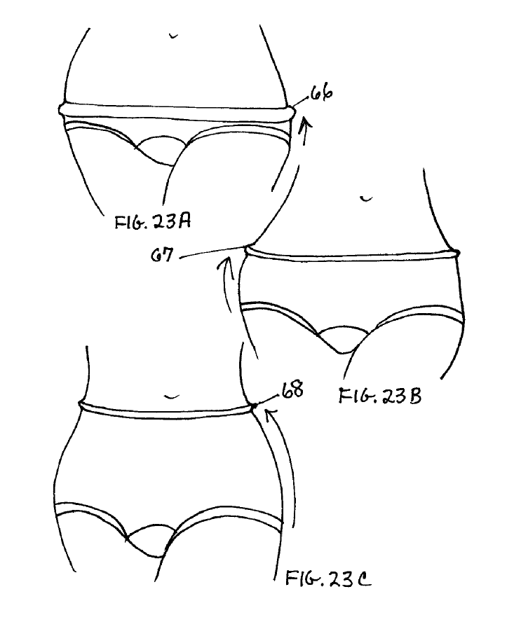

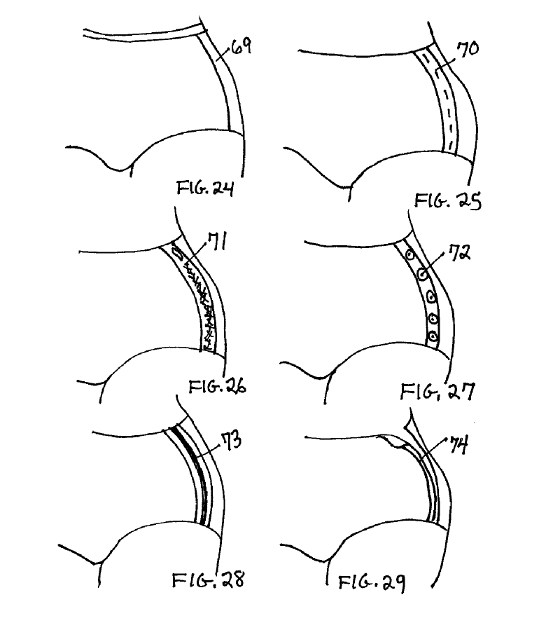

The garment device is flexible and lightweight. In one embodiment, the garment, which has been rolled up (66), is taken out of an individually wrapped, sterile package; and put on (FIG. 23a). The garment is rolled out partially (67) in (FIG. 23b) as it is put on, until it is finally completely unrolled (68) onto the body (FIG. 23c). There may be alternate ways to put on and remove the garment. The garment (FIG. 24) may have an area on one or more sides (69) for elements to facilitate fastening and removing the garment quickly and easily. Such elements may comprise, but are not limited to: perforated/pull string components (70) for easy tear off of the garment (FIG. 25), or zipper (71) component on the garment (FIG. 26), or snaps (72) on the garment (FIG. 27), or press and seal component (73) on the garment (FIG. 28), or Velcro or sticky adhesive (74) on said garment (FIG. 29). These are just a few examples, and does not limit or exclude the use of other elements, or combinations thereof by someone skilled in the art of making undergarments and/or condoms or sterile surgical gloves.

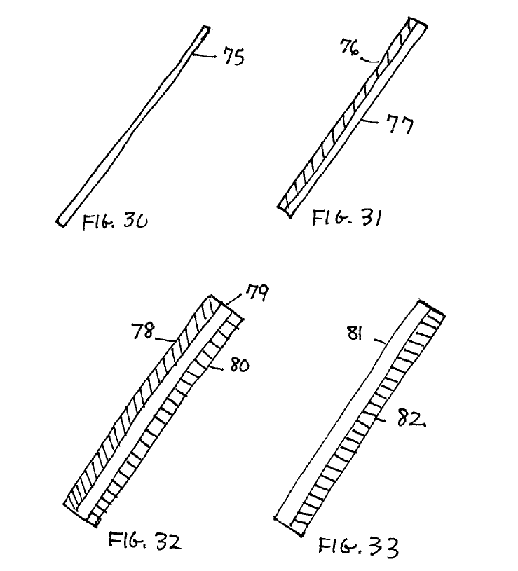

The substance material element comprising the garment itself, or any portion thereof may consist of one or more layers of substance material. In the preferred embodiment, the garment is transparent very thin, and body clinging to give the appearance of nudity. The device may be one layer thick (75) as seen in (FIG. 30). The garment, or any portion of it, may be comprised of rubber, latex, sheepskin, cellulose, nitrile, vinyl, cotton, polyester, nylon, rayon or other substance material used by someone skilled in the art. The garment, or portion thereof, should be thin, lightweight, flexible, sterile, sturdy, and fluid proof. A large portion of the garment may be composed of substance similar to sterile surgical gloves. The garment may have two layers (FIG. 31) consisting of an exterior layer (76) that could be textured, scented, and decorated, colored, or have chemical or herbal stimulant elements, and an interior layer (77) with protective barrier integral element. The garment may have three layers (FIG. 32) consisting of an exterior layer (78), with the above mentioned integral unique elements, a middle layer (79) that has an integral protective barrier shield element, and an interior layer (80) that has comfort features, since it contacts the wearer's skin Said inner layer may have absorbent wick element to absorb body moisture such as sweat, and have extra softness elements. In another embodiment, (FIG. 33), the garment has two layers of which the exterior layer (81) is the protective shield element layer, which can also have integral flavors, textures, scents, and design incorporated within said layer, and the interior layer (82) has comfort elements that contact the wearer's skin These are merely a few of the almost limitless configurations of material substance, layers, and other enhancing element features. Many other variations are to be considered within the scope and spirit of the instant invention.

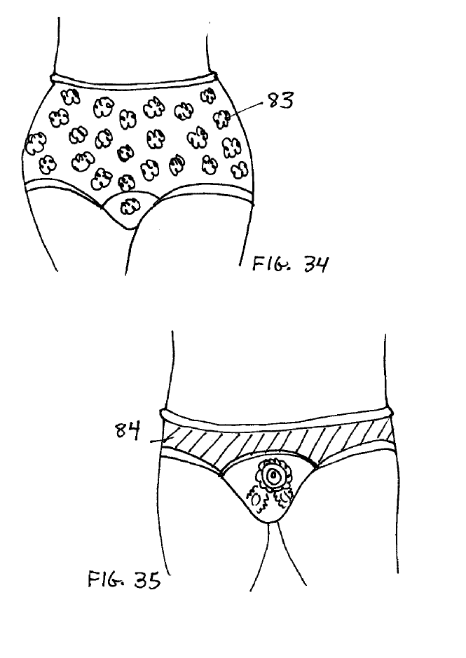

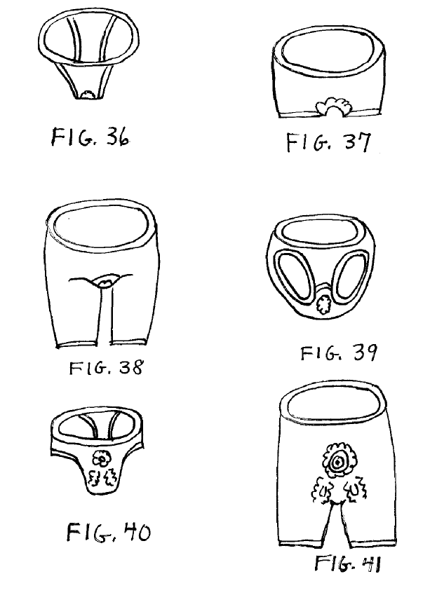

The female garment (FIG. 34) may have exterior decoration (83) with corresponding integral flavors and scents. The male garment (FIG. 35) may also have exterior decoration (84) with corresponding integral flavors and scents. Both male and female garments come in various stretchable sizes such as S, M, L, XL, etc. It is important that they fit securely to prevent leakage, and to prevent garment movement which would expose genital body surfaces. Some examples of style embodiments are: (FIG. 36) female thong, (FIG. 37) female boy cuff shorts, (FIG. 38) female bike shorts, (FIG. 39) female hi-rise, (FIG. 40) male bikini, and FIG. 41) male bike shorts.

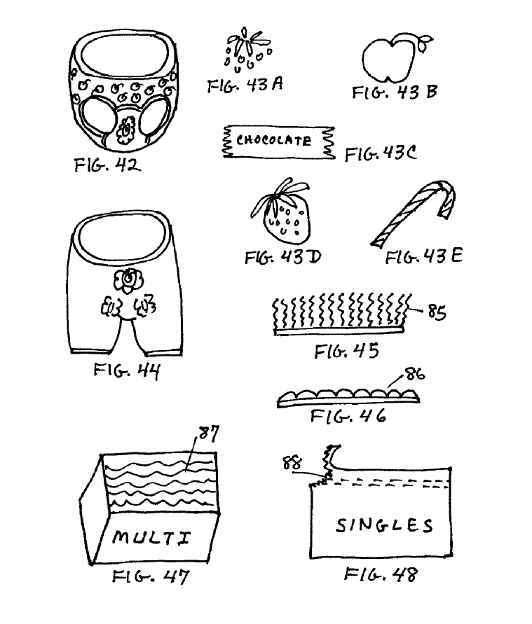

In one preferred embodiment, a female garment device (FIG. 42) may be decorated with cherry print decoration, and also have integral cherry flavor, and integral cherry scent on or within the said garment. This may act as an arousal and stimulation element and is to be considered integral to the invention. (Figures #43a-e show various decoration, flavor and scent embodiment possibilities such as: (a) vanilla, (b) apple, (c) chocolate, (d) strawberry, and (e) peppermint. Similarly, male garments (FIG. 44) may also have arousal and stimulation elements integral to the garment. Various textures may be incorporated into or onto the exterior or interior surfaces of the male and female garments. (FIG. 45) illustrates the use of a prickly texture (85), and (FIG. 46) illustrates a smoothed bumpy texture (86). Many other textures, scents, flavors that are not listed may be used, and are to be considered within the scope of the invention.

In preferred embodiments, the garments would be comprised of bio-degradable material substance; be inexpensive to manufacture, sterile, portable, and convenient. Garments could be packaged in bulk (87) as in (FIG. 47) and/or individually wrapped, with an easy tear open element (88) as in (FIG. 48), for portability and convenience. The packaging may also be disposable and bio-degradable, and may also be decorated or have colors. The full genital protective barrier garment device serves an intended purpose in an attractive way.

It should be noted that the drawings and the descriptions given are meant to convey the idea and intent of the invention, but in no way limit the scope of the invention.

What is claimed is:

1. A disposable full genital coverage protective barrier/shield undergarment device, with integral gender specific embodiments so that the device may be comfortably worn by a male or female person; which provides a prophylactic barrier/shield against, and protection from exposure to body fluids, germs, viruses, bacteria, micro-organisms, fungi, pathogens, parasites, and other contaminants, for the body area covered by, and enclosed within the boundaries of the undergarment device; and which has integral multiple sensory sexual stimulation and body enhancement component elements, and which also allows for penetration of one or more body orifices by means of integral, permanently attached, liquid impermeable, inwardly or outwardly expandable and collapsible condom/sheath component element/s, which are closed at the distal end.

2. A device as in claim #1 in which the undergarment device is comprised of component elements appropriate for accommodating either male or female genitalia, in which one embodiment is distinguished as a “male” garment, and the other embodiment is distinguished as a “female” garment; and in which said garment has at least one integral inwardly or outwardly expandable and collapsible orifice covering prophylactic condom/sheath component elements per undergarment device.

3. A device as in claim #1 in which the undergarment device protects the wearer's sexual partner from exposure to the undergarment device wearer's sperm, body fluids, germs, viruses, bacteria, micro-organisms, fungi, pathogens, parasites, and other contaminants which are covered by, and enclosed within the boundaries of the wearer's undergarment device.

4. A device as in claim #1 in which the undergarment device, in the preferred embodiment, is transparent or translucent, thin, and body clinging so as to effectuate or simulate the appearance of a nude body while wearing said undergarment device, however, in other embodiments, the fit may be looser.

5. A device as in claim #1 in which all or part of the interior and/or exterior surfaces of said undergarment device is decorated, printed, tinted, colored, embellished, textured, flavored, and scented for mutual partner sensory sexual stimulation; and said elemental embodiments are integral to the undergarment device.

6. A device as in claim #1 in which a portion of the interior, and/or exterior surfaces of said male or female undergarment device have integral textural sensory sexual stimulant elements specifically positioned proximal to the wearer's, or partner's erogenous zones or other highly sensitive skin surface areas, which may include, but is not limited to the areas of a female person's “G-spot”, “A-spot”, “U-spot”, clitoris, and anal area, and a male person's penis, scrotum, and anal area; to provide pleasure and stimulation to those areas.

7. A device as in claim #1 in which a portion of the interior, and/or exterior surfaces of said male or female undergarment device have integral herbal, natural, and or chemical sexual stimulant elements specifically positioned proximal to a wearer's, or partner's erogenous zones or other highly sensitive skin surface areas, which may include, but is not limited to the areas of a female person's “G-spot”, “A-spot”, “U-spot”, clitoris, and anal area, and a male person's penis, scrotum, and anal area; to provide pleasure and stimulation to those areas.

8. A device as in claim #1 in which the undergarment device is comprised all or in part of thin, stretchy, sturdy, liquid impermeable, liquid resistant, tear resistant sterile substance material similar to that used for sterile, disposable, surgical gloves and/or condoms.

9. A device as in claim #1 in which all, or a portion of the undergarment device may be comprised of, but is not limited to, one or more layers of substance material comprised of rubber, latex, silicon, polyurethane, sheepskin, cellulose, vinyl, nitrile, cloth, cotton, polyester, rayon, spandex, nylon, viscose, or any other substance used by someone skilled in the art of making undergarments, condoms, barrier shields, and surgical gloves, to provide comfort and protection for the wearer.

10. A device as in claim #1 which has a spermicidal component element integral within the substance material, and/or as an interior or exterior element applied to the surface of a condom component element on a male or a female undergarment device.

11. A device as in claim #1 which has a lubricant component element integral within the substance material of the undergarment device, and/or which has a lubricant element integrally applied to the interior and/or exterior surface/s of a condom component element of the male and/or female undergarment device; and said lubricant may contain sexual stimulant and/or sensation enhancement elements.

12. A device as in claim #1 in which the undergarment device fits snugly upon the wearer's body contours, and has secure leg bands and a secure waistband to help prevent leakage of fluids, and shifting of the undergarment; which might decrease the level of protection.

13. A device as in claim #1 in which the undergarment has elements such as snaps, adhesive, Velcro, press and seal element, perforated tear element, zippers, or other elements on the sides, or other areas of the undergarment to facilitate easy donning and removal of said garment; as well as other elements which are integral to the substance material of the device which make the device easy to slide on and off.

14. A device as in claim #1 in which the male and/or female undergarment device has an integral back seam element between the buttock cheeks; to define and enhance the appearance of the buttocks of the wearer of the garment.

15. A device as in claim #1 in which the male and/or female undergarment device has integral padding, molding or contour element on the garment buttocks area, or other genital area; to give the appearance of larger buttocks, and/or larger genitals of the wearer of the garment, or to otherwise enhance the genital area.

16. A device as in claim #1 in which the male undergarment device has stretchy integral optimal body temperature control and retention element, and softness and absorption comfort elements integral within the material fabric substance that is proximal to the scrotum and testicle area of the device.

17. A device as in claim #1 in which the integral, liquid impermeable, stretchy, smooth or textured, ringed, bellowed, folded, pleated, scrunched, telescoped, or rolled, inwardly expanding/extending component element which covers the vaginal vestibule area; and which may be compressed into a flattened position while in the un-extended or un-expanded state, comprises a female prophylactic condom; which is integral and proximal to the mid crotch area of said “female” undergarment device.

18. A device as in claim #1, in another embodiment, in which the integral, liquid impermeable, stretchy, smooth or textured, ringed, bellowed, folded, pleated, scrunched, telescoped, or rolled, outwardly expanding/extending component element which covers the penis, and has a reservoir tip at the distal end; and which may be compressed into a flattened position while in the un-extended, un-expanded state, comprises a male prophylactic condom; which is integral and proximal to the upper crotch area of said “male” undergarment device.

19. A device as in claim #1, in another embodiment, in which another integral, liquid impermeable, stretchy, smooth or textured, ringed, bellowed, folded, pleated, scrunched, telescoped, or rolled, inwardly or outwardly expanding/extending component element which covers the anal orifice area, and which may be compressed into a flattened position while in the un-extended or un-expanded state; comprises an anal unisex prophylactic condom which is integral and proximal to the rear crotch area of the “male” undergarment device, and is integral and proximal to the rear crotch area of the “female” undergarment device; in which said anal unisex prophylactic condom is in addition to the female condom, and is in addition to the male condom on said undergarment device.

20. A device as in claim #1 in which the undergarment device is comfortable, portable, preferably bio-degradable, inexpensive, and comes in a variety of sizes, and styles which may include, but is not limited to: g-string, bikini, panty, brief, boy cuff shorts, biker shorts, hi-rise, and thong; and may be packaged in bulk, or individually wrapped; and is appealing to those persons interested in wearing an attractive safe sex undergarment device that does not have the look or stigma of an adult diaper.
